(19) United States
(12) Patent Application Publication
(10) Pub. No.: US 2005/0123491 A1
(43) Pub. Date: Jun. 9, 2005

(54) Composition and method for treating onychomycosis

(76) Inventor: Michael G. Petit, Montecito, CA (US)

Correspondence Address: Michael G. Petit, P.O. Box 91929, Santa Barbara, CA, 93190-1929, US

(21) Appl. No.: 10/805,463

(22) Filed: Mar. 19, 2004

Related U.S. Application Data
(60) Provisional application No. 60/528,178, filed on Dec. 8, 2003.

(51) Int. Cl. A61K 7/04; A61K 31/785; A61K 31/045

(52) U.S. Cl. 424/61; 424/78.3; 514/724

(57) ABSTRACT

A fluid composition and a method for treating fungal infections of the nail. The composition includes essentially polyvinylpyrrolidone (PVP) and a low molecular weight alcohol in aqueous solution. A first preferred composition is comprised of ethanol in a polyvinylpyrrolidone hydrogel. The first preferred composition contains equal parts (w/w) of PVP (MW˜55,000) and aqueous ethanol (20-80% ethanol). The compositions may further include antifungal agents other than an alcohol. In accordance with the method, the infected nail(s) is clipped and the upper surface of the nail is abraded with an emery board or sandpaper and the viscous composition applied to the abraded upper surface of the nail and under the free distal end of the nail and allowed to dry to form a hard, hydrophilic layer on the nail (˜1 hour). The composition is reapplied to the infected nail once each day.

### REFERENCE TO RELATED APPLICATIONS

This application claims the benefit of U.S. Provisional Application No. 60/528,178, filed Dec. 8,2003, entitled: “Composition and Method for Treating Onychomycosis”.

### BACKGROUND OF THE INVENTION

1. Field of the Invention

The present invention relates to an antifungal composition and a method for using the composition for the treatment of onychomycosis.

2. Prior Art

Onychomycosis, also called ringworm of the nails, or tinea unguium, is a fungus infection of the nails causing thickening, roughness and splitting. The infection is caused by various fungal species including Trichophyton rubrum, Trichophyton tonsurans, Epidermophytonfloccosum and Trichophyton mentagrophytes. Once these microorganisms establish themselves within or under a nail, eradication with current over-the-counter (OTC) antifungal agents is difficult, costly and time consuming and recurrences of the disease can be expected. Antifungal agents that are formulated for topical application, such as undecylenic acid, lotrimin and tolnaftate, are effective for treating fungal infections of the skin but, as presently formulated and administered, are ineffective for treating fungal infections of the nail.

Current treatment regimens for onychomycosis include prolonged and sustained application of topical fungicidal creams and/or solutions directly to the infected nail(s) and/or systemic treatment with antifungal drugs such as griseoflulvin, terbinafine, and itraconazole. Some of the systemic treatments have undesirable side effects such as nausea, headache, photosensitivity, gastrointestinal intolerance, elevated liver enzymes, and undesirable drug interactions, making the value of the treatment questionable.

To overcome certain of the problems associated with prior art therapeutic regimens for onychomycosis, the present inventor, in copending U.S. patent application Ser. No. 10/370,129, filed Feb. 18, 2003, discloses a drug-dispensing dressing for administering a fluid containing an antifungal agent to a fungus infected nail on a toe or a finger. In a preferred embodiment, the dressing includes: (a) a reservoir comprising a substantially flat, fluid-absorbent pad having a first hole therein; and (b) an elongate strip of a fluid impermeable material having a central portion with a second hole therein. The elongate strip has a skin-facing surface coated with a layer of adhesive. The skin-facing surface of the central portion of the strip is affixed to the fluid-absorbent pad with the second hole overlying the first hole. The dressing is affixed to a toe and the reservoir, which is rechargeable, is charged with a fluid antifungal agent introduced dropwise or injected through the hole where the fluid is absorbed and disbursed by the pad and slowly released to the underlying nail. The dressing may further include a removable, preferably elastic, barrier layer disposed over the holes. In another embodiment, the reservoir is attached to the inner surface of an elastic tube to underlie a hole in the tube. The tubular dressing is stretched and placed over the affected toe with the reservoir overlying the infected nail and the antifungal agent introduced into the reservoir via the hole.

Lorenz, in U.S. Pat. Nos. 5,156,601; 5,258,421 and 5,306,504 discloses the use of compositions containing polyvinylpyrrolidone (PVP) and a urethane. Lorenz discloses that such compositions can be used as a carrier for certain antibiotic compositions, including antifungal compositions. The disclosed compositions are “tacky” and form an adhesive surface when applied to the skin. Artisans skilled in the art will recognize that the treatment of onychomycosis is far more difficult than the treatment of dermatophytes infecting the skin. For example, it is extremely difficult to deliver prior art antifungal compositions that are operable for treating fungal infections of the skin, into an infected nail by topical application thereto. There is a continuing need for improved antifungal compositions, and methods for using such compositions, for the treatment of onychomycosis.

### SUMMARY

It is an object of the present invention to provide a fluid antifungal composition for topical application to a nail for the treatment of onychomycosis.

It is a further object of the invention to provide a fluid antifungal composition meeting the above objective that forms a hard dry layer on the infected nail following application thereto for the treatment of onychomycosis.

The above objectives are met, in part, by the provision of an antifungal composition for topical application to a nail infected with a fungus, the topically applied composition thereafter being effective for treating the infected nail. The antifungal composition comprises essentially a viscous solution comprising polyvinylpyrrolidone (PVP) and an alcohol, preferably ethanol. The alcohol preferably contains four or less carbon atoms.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a longitudinal cross-sectional view of a nail attached to a toe showing the anatomical relationship between the nail and the supporting structure.

### DESCRIPTION OF THE PREFERRED EMBODIMENTS

The terms “antimycotic agent” and “antifungal agent” are used interchangeably herein to mean any composition that either kills or inhibits the growth of a pathogenic fungus infecting the nail of a digit.

The term “pathogenic fungi”, as used herein, includes, but is not limited to, the fungal organisms most commonly associated with onychomycosis such as T. rubrum and T. mentagrophytes.

The term “fungicidal agent”, as used herein, means a therapeutic agent that kills pathogenic fungi and their spores.

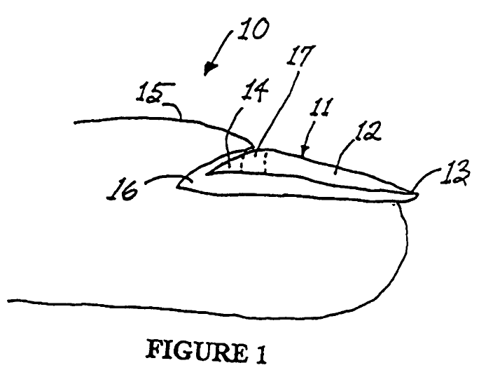

With reference now to FIG. 1, a toe 10 comprises a nail or unguis 11 having a nail body 12, a free edge 13 and a nail root 14. The nail body 12 is a horny, keratinized material referred to as the stratum lucidum. The root 14 fastens the nail 11 to a finger or toe by fitting into a groove in the skin 15. A nail matrix (germinal layer) 16 lies beneath and posterior to the nail body 12 and the nail root 14. The nail matrix 16 is vascularized and firmly connects the nail to the underlying connective tissue. The nail 11 grows in length by proliferation of germ cells in the nail root 14 and grows thicker by proliferation of cells in the portion of the nail matrix beneath the lunula 17. The site of fungal infection in the nail may be limited to the horny, keratinized nail body 12 or it may further involve the nail matrix 16. In most cases of onychmycosis, the fungal mycelium penetrates and infects the tough stratum lucidum and it is difficult to deliver a therapeutically effective dosage of an antifungal agent to the site of infection. Since there is aqueous fluid in the proteinaceous matrix comprising the nail body, a hydrophobic antifungal agent comprised of, for example, an oil such as lavender or tea tree oil, may not be able to penetrate the stratum lucidum and, either alone or in combination with a compatible antifungal agent, provide a therapeutic composition effective for treating onychomycosis. Accordingly, a new class of hydrophilic, dessicant antifungal compositions has been developed which, when applied to a properly prepared nail surface, enhances migration of water out of an infected nail and/or the delivery of an antifungal agent into the nail.

Antifungal Compositions

A fluid composition comprised of polyvinylpyrrolidone (PVP) in water is a clear, viscous gel that may be made in a variety of viscosities, all of which are miscible with ethyl and isopropyl alcohol. Antifungal agents such as ethanol contained within the PVP/water composition are slowly absorbed by a nail, particularly when the exposed upper surface of the nail is abraded (prior to the application of the aforesaid mixture to the nail) to present a roughened surface. The PVP acts as a dessicant and can be employed as a carrier and permeation enhancer for the delivery of an antifungal agent such as, for example, ethanol or isopropanol, into the nail.

A fluid composition comprised essentially of a PVP hydrogel and either ethanol or isopropanol is a particularly preferred composition for the topical treatment of onychomycosis when administered to an infected nail in accordance with the method that will be described below. The particularly preferred fluid antifungal composition is an aqueous hydrogel comprising essentially 20-60% (by weight) of PVP (Sigma-Aldrich) having a molecular weight between 15,000 and 150,000, and a water-soluble antifungal agent, preferably ethyl or isopropyl alcohol. The fluid composition may further include other water-soluble antifungal agents.

Method for Treating Onychomycosis

The digit bearing a nail having a mycotic infection (i.e., the infected nail) is presented and the nail is clipped. Burs are removed from the clipped end of the nail with an emery board. The nail and supporting digit are then cleaned with isopropyl alcohol and the upper, exposed surface of the nail is abraded to create a roughened surface. A layer of a fluid antifungal composition comprising an alcohol in an aqueous solution containing PVP is then applied to the roughened surface of the nail by applicator means such as a syringe, a tubular dispenser, a brush, a rod or a spatula. The layer of antifungal composition deposited on the infected nail is permitted to air-dry until the layer hardens to a glassy surface (˜1 hour for a viscous solution of PVP in aqueous ethanol (50% w/w)). If the digit is a toe, when the glassy layer is dry, a sock may be donned. The antifungal composition is easily removed from the infected nail with water. After removal of the antifungal composition, the infected nail is dried, preferably with a hair dryer, re-abraded and re-coated with a film of the antifungal composition. Three to seven weeks of treatment is required to cure most cases of onychomycosis in accordance with the present composition and method, although the discoloration of the nail associated with separation of the nail from the nail bed may persist for several months thereafter.

While particular embodiments of the present invention have been illustrated and described, it would be obvious to those skilled in the art that various other changes and modifications can be made without departing from the spirit and scope of the invention. For example, a solution containing 50% PVP, 20% water and 30% isopropyl alcohol or another antifumgal alcohol such as ethyl alcohol also provides an effective therapeutic antifungal composition. Small alcohols having 1-4 carbon atoms are preferred antifungal agents for use with a PVP composition in accordance with the present invention. The choice of the low molecular weight alcohol does not appear to be critical. For example, solutions containing 50% (w/w) PVP in both 50% and 75% aqueous ethanol were found to be efficacious for treating onychomycosis. It is therefore intended to cover in the appended claims all such changes and modifications that are within the scope of this invention.

1. A fluid antifungal composition for topical application to a nail infected with a fungus, said topically applied composition thereafter being effective for treating said infected nail, said antifungal composition comprising essentially a solution containing polyvinylpyrrolidone in water.

2. The antifungal composition of claim 1 further comprising 1-40% (w/w) ethanol.

3. The antifungal composition of claim 1 further comprising 1-40% (w/w) isopropyl alcohol.

4. A method for treating onychomycosis comprising the application of a layer of a fluid antifungal composition in accordance with claim 1 to a nail infected by a fungal organism.

5. A method for treating onychomycosis comprising the application of a layer of a fluid antifungal composition in accordance with claim 2 to a nail infected by a fungal organism.

6. A method for treating onychomycosis comprising the steps of: (a) presenting a digit having an infected nail thereon; (b) clipping a distal end of the infected nail; (c) abrading an exposed upper surface of the infected nail; and (d) applying a layer of a fluid antifungal composition to the abraded upper surface of the infected nail.

7. The method for treating onychomycosis of claim 6 wherein the fluid antifungal composition comprises polyvinylpyrrolidone and an alcohol containing 1-4 carbon atoms.
